(19) United States
(12) Patent Application Publication
(10) Pub. No.: US 2024/0206732 A1
(43) Pub. Date: Jun. 27, 2024

(54) SMART GLASSES, DEVICE AND METHOD FOR HEALTH INFORMATION NOTIFICATION

(71) Applicant: SOLOS TECHNOLOGY (SHENZHEN) LIMITED, Shenzhen (CN)

(72) Inventors: Mei Sze Au-Yeung, Shenzhen (CN); Chiu Ming So, Shenzhen (CN); Wai Kuen Cheung, Shenzhen (CN)

(21) Appl. No.: 18/594,114

(22) Filed: Mar. 4, 2024

Related U.S. Application Data
(63) Continuation of application No. PCT/CN2022/114822, filed on Aug. 25, 2022.

(30) Foreign Application Priority Data
Sep. 3, 2021 (CN) 202111031945.6

(51) Int. Cl. A61B 5/00; A61B 5/01; A61B 5/11

(52) U.S. Cl. CPC A61B 5/0008; A61B 5/0022; A61B 5/01; A61B 5/1112; A61B 5/6803; A61B 5/746; A61B 2560/0252; A61B 2562/0271; A61B 2562/04

(57) ABSTRACT

Disclosed are smart glasses, device and method for health information notification. The smart glasses include a glasses body and a plurality of temperature sensors, and/or UV sensors, and/or external temperature sensors, wherein the temperature sensors are provided on an inner side of the glasses body, with a distance from a contact site of user's skin being less than a preset distance. According to the method for health information notification, whether user's body temperature is abnormal is determined based on body temperature data measured by the smart glasses, and the user is prompted in time if the body temperature is abnormal. The smart glasses, device and method for health information notification can improve the accuracy of body temperature measurement, and improve the accuracy of health information notifications for users.

This application is a Continuation Application of PCT Application No. PCT/CN2022/114822, filed Aug. 25, 2022, which claims the priority of Chinese patent application No. 202111031945.6, entitled “smart glasses, device and method for health information notification” and submitted to the State Intellectual Property Office of P.R.C on Sep. 3, 2021, the entire contents of which are hereby incorporated by reference.

### TECHNICAL FIELD

Embodiments of this application relates to the technical field of smart wearables, and in particular to smart glasses, device and method for health information notification.

### BACKGROUND

With the development of smart devices, smart wearables are used to measure motion parameters, biological parameters, environmental parameters, and etc. by users, and measurement results can be obtained by the users through viewing interfaces of the smart wearables.

Biological parameters are important parameters reflecting human health. For biological parameters of the human body, the most common manner is to use a smartwatch to measure heart rate, blood pressure, respiration, body temperature, blood oxygen saturation, and etc. Among them, when measuring the body temperature, due to the fact that the temperature of the human wrist is easily affected by accidental factors, such as hand washing, there may be a deviation in the body temperature measured by the smartwatch, which may affect the accuracy of health information notifications for users.

### SUMMARY

Embodiments of this application provide smart glasses, device and method for health information notification, which can solve the problem of temperature measurement deviation caused by accidental factors and thus affecting the accuracy of health information notifications for users.

On one hand, an embodiment of this application provides a pair of smart glasses, including:
a glasses body and a plurality of temperature sensors;
the temperature sensors being provided on an inner side of the glasses body, with a distance from a contact site of user's skin being less than a preset distance.

Further, the temperature sensors are set at one or multiple positions of inner sides of temples of the glasses body, an inner side of an upper edge of a glasses frame of the glasses body, and an inner side of a lower edge of the glasses frame of the glasses body.

Further, the temperature sensors are used to measure user's body temperature and/or ambient temperature around the user.

Further, the smart glasses further include at least one UV sensor being set at one or multiple positions of an outer side of a glasses frame of the glasses body and outer sides of temples of the glasses body.

Further, the at least one UV sensor includes a plurality of UV sensors arranged on an upper face of the glasses frame.

Further, the smart glasses further include a plurality of external temperature sensors being set at one or multiple positions of an outer side of a glasses frame of the glasses body and outer sides of temples of the glasses body.

Further, the external temperature sensors are used to measure an ambient temperature around the user.

On another hand, an embodiment of this application provides a method for health information notification, applied to a pair of smart glasses which include a glasses body, and a temperature sensor and a processor provided on the glasses body, the processor being electrically coupled with the temperature sensor, the method including:
obtaining body temperature data measured by the temperature sensor through the processor;
determining whether user's body temperature is abnormal through the processor based on the body temperature data; and
outputting a notification that user's body temperature is abnormal through the processor when the user's body temperature is abnormal.

On another hand, an embodiment of this application provides a device for health information notification, including:
a memory for storing computer programs; and
a processor coupled with the memory, the processor being configured for calling and executing the computer programs stored on the memory;
the computer programs including:
an acquisition instruction for obtaining body temperature data measured by a temperature sensor set on a glasses body of a pair of smart glasses;
a judgment instruction for determining whether user's body temperature is abnormal based on the body temperature data;
a notification instruction for sending a notification that user's body temperature is abnormal when the user's body temperature is determined to be abnormal.

From the above embodiments of this application, it can be seen that the smart glasses include a glasses body and a plurality of temperature sensors, wherein the temperature sensors are provided on an inner side of the glasses body, with a distance from a contact site of user's skin being less than a preset distance. As the facial temperature of the human body is less affected by external factors, and the temperature sensors are set closer to the skin, it is easy to obtain more accurate body temperature and thus can improve the accuracy of body temperature measurement.

### BRIEF DESCRIPTION OF THE DRAWINGS

In order to illustrate embodiments of this application or the technical solutions in the prior art more clearly, drawings that need to be used in the description of the embodiments or the prior art will be briefly described below. It is obvious that the drawings described below are only some embodiments of this application. For those skilled in the art, other drawings can be obtained based on these drawings without any creative work.

FIG. 1 is a schematic view of a pair of smart glasses provided by an embodiment of this application.

FIG. 2 is a schematic view of a pair of smart glasses provided by another embodiment of this application.

FIG. 3 is a flowchart of a method for health information notification provided by an embodiment of this application.

FIG. 4 is a flowchart of a method for health information notification provided by another embodiment of this application.

FIG. 5 shows a corresponding relationship curve between indoor temperature values and measurement time in a method for health information notification provided by another embodiment of this application.

FIG. 6 is a schematic view of a device for health information notification provided by an embodiment of this application.

### DESCRIPTION OF THE EMBODIMENTS

For better illustrating and understanding the purpose, features and advantages of the present application, detailed description will be given for the embodiments provided by the present application with reference to the append drawings. Obviously, the described embodiments are only a part of the embodiments, and not all of the embodiments of this application. Based on the embodiments of this application, all other embodiments obtained by those skilled in the art without any creative work should be in the scope of this application.

An embodiment of this application provides a pair of smart glasses, wherein a glasses body of the smart glasses is provided with a plurality of temperature sensors that can measure user's body temperature. The temperature sensors is set on an inner side of the glasses body, with a distance from a contact site of user's skin being less than a preset distance. That is, the temperature sensor is set at the inner side of the glasses body and located at a position close to user's skin, so as to ensure the accuracy of body temperature measurement. Further, the temperature sensor may measure an ambient temperature around the user. That is, the temperature sensor is used to measure user's body temperature and/or ambient temperature.

Specifically, the plurality of temperature sensors may be set at one or multiple positions of inner sides of temples of the glasses body, an inner side of an upper edge of a glasses frame of the glasses body, and an inner side of a lower edge of the glasses frame of the glasses body, wherein multiple positions mean two or more positions. The temperature sensor(s) may be one or more. When the temperature sensors are more than one, they may be evenly or unevenly distributed in the multiple positions mentioned above.

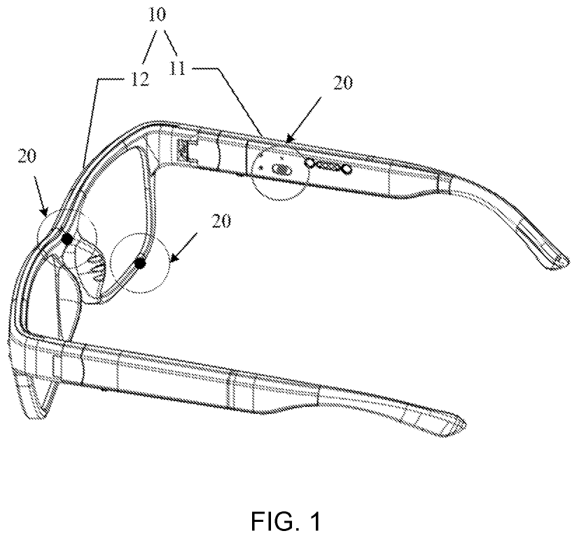

Referring to FIG. 1, which is a schematic view of a pair of smart glasses provided by an embodiment of this application. For easy of illustration, only parts related to this embodiment of the present application are shown. As shown in FIG. 1, the smart glasses include a glasses body 10 and a plurality of temperature sensors 20, wherein the glasses body includes temples 11 and a glasses frame 12. The temperature sensors 20 are set on inner sides of the temples 11 of the glasses body 10, an inner side of an upper edge of the glasses frame 12 of the glasses body 10, and an inner side of a lower edge of the glasses frame 12 of the glasses body 10, respectively. FIG. 1 exemplarily shows positions of three temperature sensors 20. Any number of temperature sensors 20 set on other positions of the temples 11, and on other positions of the glasses frame 12 should be also within the protection scope of this application.

In this embodiment of the present application, the smart glasses include a glasses body and a plurality of temperature sensors. The temperature sensor is set on an inner side of the glasses body, with a distance from a contact site of user's skin being less than a preset distance. As the facial temperature of the human body is less affected by external factors, and the temperature sensor is set closer to the skin, it is easy to obtain more accurate body temperature and thus can improve the accuracy of body temperature measurement.

In another embodiment of this application, the smart glasses further include a plurality of ultraviolet (UV) sensors for measuring UV content around the human body. The UV sensors are set at one or multiple positions of an outer side of the glasses frame of the glasses body, and outer sides of temples of the glasses body, which can better receive sunlight and thus the measured UV values are more accurate. The UV sensor(s) may be one or more. When the UV sensors are more than one, they may be evenly or unevenly distributed in the multiple positions mentioned above. Specifically, the plurality of UV sensors is set on an upper face of the glasses frame 12.

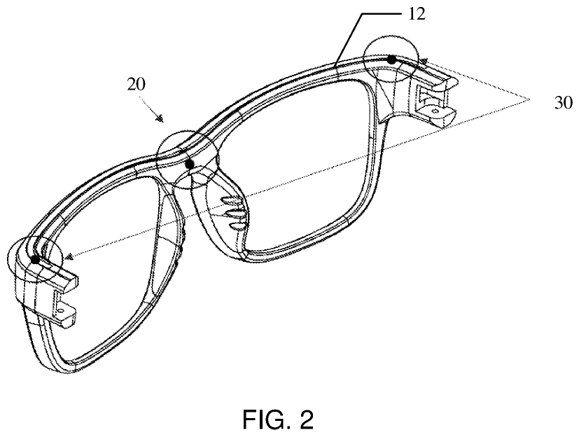

Referring to FIG. 2, which is a schematic view of a pair of smart glasses provided by another embodiment of this application. In FIG. 2, two UV sensors are taken as an example. The two UV sensors 30 are set on two lateral sides of the upper face of the glasses frame 12, respectively. In FIG. 2, a temperature sensor 20 is provided at an inner side of a middle of an upper edge of the glasses frame 12. It should be noted that FIG. 2 is just an example. In the embodiments of this application, several temperature sensors and several UV sensors may be respectively or simultaneously provided on the smart glasses.

In this embodiment of the present application, the smart glasses include a glasses body, a plurality of temperature sensors, and a plurality of UV sensors. The temperature sensors are set at one or multiple positions of inner sides of temples of the glasses body, an inner side of an upper edge of a glasses frame of the glasses body, and an inner side of a lower edge of the glasses frame of the glasses body. The UV sensors are set at one or multiple positions of an outer side of the glasses frame of the glasses body and outer sides of the temples of the glasses body, with less head obstruction and easier absorption of sunlight, so that the measurement of UV content is accurate. The smart glasses can simultaneously measure body temperature, outdoor temperature, and UV content of the environment, with accurate and comprehensive measurement values, providing more accurate basis for evaluating internal and external factors of human health, and improving the practicality and intelligence of the smart glasses.

Further, indoor temperature measured by the temperature sensor is the temperature of the integrated circuit (IC) of the temperature sensor. After the user wears the smart glasses for a period of time, the temperature change of the head will affect the temperature of the smart glasses, causing the temperature of the IC of the temperature sensor to rise, resulting in a continuous increase in the measured indoor temperature. It is estimated that this cannot accurately reflect the actual indoor temperature, leading to an impact on the measured body temperature.

In another embodiment, in order to further improve the accuracy of the smart glasses in measuring body temperature and ambient temperature, the smart glasses further include a plurality of external temperature sensors. The external temperature sensors are set at one or multiple positions of an outer side of the glasses frame 12 and outer sides of the temples 11, for measuring the ambient temperature around the user.

In this embodiment of the present application, external temperature sensors are provided on the smart glasses, which are not close to the head and will less affected by the head temperature. Compared to the temperature sensors close to the head, the external temperature sensors can measure the ambient temperature and body temperature more accurately.

An embodiment of this application further provides a method for health information notification based on the smart glasses of the above embodiments, which will be described in detail below.

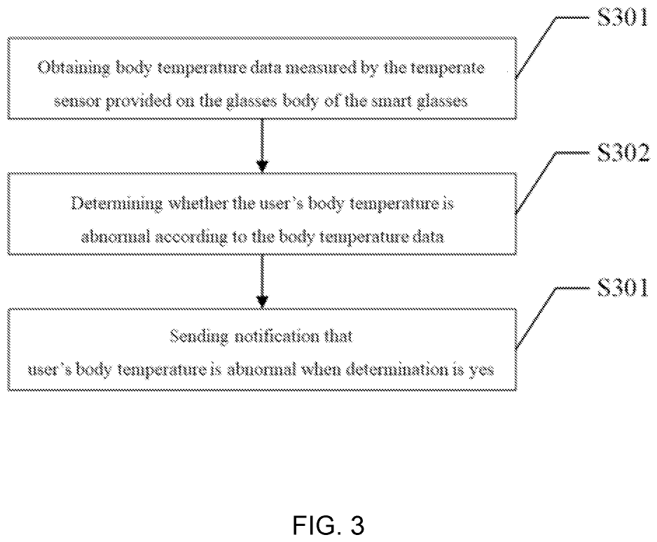

Refer to FIG. 3, which shows a flowchart of a method for health information notification provided by an embodiment of this application. This method for health information notification is applied to the smart glasses of the above embodiments. The smart glasses include a glasses body, and a temperature sensor and a processor provided on the glasses body, wherein the processor is electrically coupled with the temperature sensor. An execution subject may be the processor of smart glasses, and the method includes:
S301, obtaining body temperature data measured by the temperature sensor through the processor;
the processor of smart glasses obtains user's body temperature data measured by each of the temperature sensors.
S302, determining whether user's body temperature is abnormal through the processor based on the body temperature data;
determining manner may bedetermined by calculating an average body temperature: counting all of the body temperature data measured during a preset period, removing invalid body temperature data higher than a preset maximum limit or lower than a preset minimum limit, and calculating an average value of remaining effective body temperature data. This average value is the user's body temperature. If the average value exceeds a preset normal range, it is confirmed that the user's body temperature is abnormal.

For example, the preset period may be 1 minute, the maximum limit value may be 45° C., the minimum limit value may be 30° C., and the normal range may be 36˜37° C.

Calculating manner may also be determined by counting a number of abnormal body temperatures: counting all of the body temperature data within the preset period. If the number of times the body temperature data exceeds the preset normal range is greater than a preset number, it is confirmed that the user's body temperature is abnormal.

For example, the preset number of times may be 3.

Abnormal body temperature may refer to abnormally high or abnormally low body temperature.

Further, the ambient temperature value measured by the temperature sensor can affect the determination of body temperature. This temperature sensor may generate a measurement deviation as the temperature of the human body rises. To correct this deviation, an external temperature sensor may be used to measure more accurate ambient temperature value. Specifically, obtaining ambient temperature value measured by the external temperature sensor set out of the glasses body, and comparing it with the ambient temperature value measured by the temperature sensor provided on the glasses body. If the comparison result is inconsistent, i.e., a difference between the ambient temperature values measured by the external temperature sensor and the temperature sensor on the glasses body is greater than a preset temperature difference, it is determined that the ambient temperature value measured by the temperature sensor on the glasses body is inaccurate. At this point, the ambient temperature value measured by the external temperature sensor is taken as the actual ambient temperature value, which improves the accuracy of ambient temperature measurement.

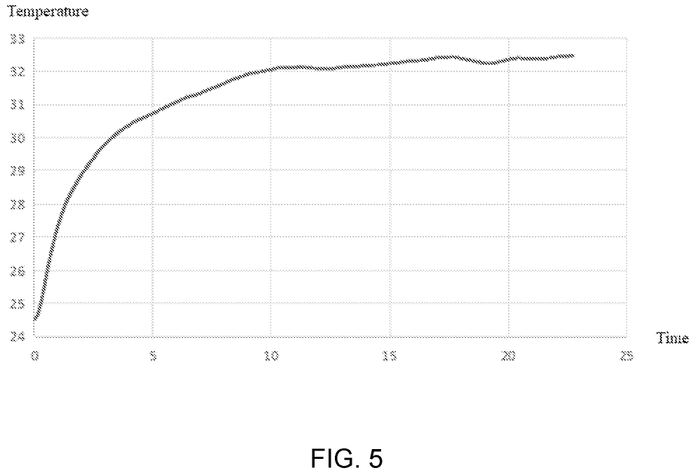

Furthermore, it is also possible to determine whether the current indoor temperature has truly changed by learning the indoor temperature change curve after the user wearing the smart glasses for a long time, thereby determining the true value of the user's body temperature. Specifically, based on the measured indoor temperature values and measurement time of the temperature sensor, a corresponding relationship curve between the indoor temperature values and the measurement time is generated and saved. An example of this corresponding relationship curve can be seen in FIG. 5. When the indoor temperature value measured by the temperature sensor continues to rise, the raised indoor temperature value and measurement time are compared with the corresponding relationship curve. If the raised indoor temperature value and measurement time match the corresponding relationship curve, that is, a difference between the raised indoor temperature value and measurement time and the corresponding relationship curve meets a preset standard, such as a distance between the point corresponding to the raised indoor temperature value and measurement time and the corresponding relationship curve is less than a preset distance, it indicates that the current indoor temperature rise is caused by the temperature rise of the user's head. Therefore, based on the corresponding relationship curve, search for the historical body temperature value of the user before the temperature rises, and determine this historical body temperature value as the user's current true body temperature value. If the raised indoor temperature value and measurement time do not match the corresponding relationship curve, it indicates that the current indoor temperature rise is indeed a real rise, for example, the user walked from indoors to outdoors, and the current measured temperature value of the user will be used as the user's current real temperature value. It can more accurately determine the user's true body temperature value and improve the accuracy of the smart glasses in body temperature measurement.

Further, the user's body temperature values and/or UV values in the environment are uploaded to a server, so that the server can send the current body temperature value and historical body temperature value of the user, and/or, the current UV value and historical UV value in the environment where the user is located to the client for display based on the user's query operation through the client (APP, application), improving the convenience of user operations.

Alternatively, the user's body temperature values and/or UV values in the environment are uploaded to a server (such as a cloud server), so that the server analyzes whether the uploaded body temperature values within a preset first analysis cycle exceed a preset first body temperature reference value, and/or, whether the uploaded UV values within the preset first analysis cycle exceed a preset first UV reference value; or, whether the uploaded body temperature values within a preset second analysis cycle exceed a preset second body temperature reference value, and/or, whether the uploaded UV values within the preset second analysis cycle exceed a preset second UV reference value, wherein the first body temperature reference value is greater than the second body temperature reference value, and the first UV reference value is greater than the second UV reference value. Specifically, the first body temperature reference value may be a reference value corresponding to a sum of the body temperature value in the preset first analysis cycle, and the second body temperature reference value may be a reference value of the body temperature value in the preset second analysis cycle; the first UV reference value may be a reference value corresponding to a sum of the UV values in the preset first analysis cycle, and the second UV reference value may be a reference value of the UV values in the preset second analysis cycle. Taking the UV values as an example, for example, the first analysis cycle is one month, and the second analysis cycle is one day. Correspondingly, long term analysis of the UV values that the user is exposed to may include analyzing whether the accumulated UV values in one month exceeds the first UV reference value, that is, whether the total UV values that the user is exposed to in that month is too high. It may also analyzes whether there are UV values that exceed the second UV reference value within one day, that is, whether there are extreme UV values that the user is exposed to in that day. This can further achieve the intelligence and convenience of body temperature and UV value analysis.

During the first analysis cycle, if the body temperature values exceed the preset first body temperature reference value, and/or, the UV values exceed the preset first UV reference value; or, during the second analysis cycle, if the body temperature values exceed the preset second body temperature reference value, and/or, if the UV values exceed the preset second UV reference value, a health warning information and response information will be sent to the client. The health warning information is used to remind the user that he/she may experience heatstroke, and the response information is used to prompt the user to enter the room, rest in a cool place, and drink water.

Alternatively, the UV values in user environment may be uploaded to a server (such as a cloud server), and according to a preset sending cycle or based on the user's query operation through the client, the historical UV values exceeding the preset UV reference value within the preset cycle may be marked and sent to the client for display.

Among them, the historical UV values exceeding the preset UV reference value is recognized by the processor of the smart glasses as an excessively high UV value, which can be used for long-term analysis of the user's UV exposure data. Specifically, the preset UV reference value may include a preset first UV reference value and a preset second UV reference value, wherein the first UV reference value is greater than the second UV reference value. Specifically, the first UV reference value may be a reference value corresponding to a sum of UV values within the preset first analysis cycle, and the second UV reference value may be a reference value of UV values within the preset second analysis cycle. This can further achieve the intelligence and convenience of UV value analysis.

Marking may be to mark the UV values that exceed the preset UV reference value in a format different from that do not exceed the preset UV reference value in font color, thickness, or size, or mark the sum of UV values within the first analysis cycle and/or the UV values within the second analysis cycle in the form of a generated chart, wherein a purpose is to provide users with more intuitive analysis results of UV values in the environment.

Furthermore, big data analysis may be used to provide analysis and notification on heatstroke risk for populations on a regional basis. The region may be divided based on geographical area, population, or geographical characteristics, such as a region with similar geographical area, population, or geographical characteristics, or may be divided according to the current administrative region.

Confirm the user's current location and upload the measured body temperature values and/or UV values in the environment to a server (such as a cloud server), so that the server can use a preset big data analysis method to determine that: among the preset population in the region, whether a number of people whose body temperature exceeds a preset body temperature warning value exceeds a preset number of people, and/or, whether a number of people whose UV value exceeds a preset UV warning value exceeds a preset number of people. The preset population may be set according to age, gender, or physical condition, such as young children, women, or those whose physical examination data shows weakness. Among the preset population in the region, if the number of people whose body temperature exceeds the preset body temperature warning value exceeds the preset number of people, and/or, if the number of people whose UV value exceeds the preset UV warning value exceeds the preset number of people, it indicates that there are more people at risk of heatstroke among the preset population in the region, and health warning message and response message will be sent to the client to remind the user that there is a high risk of heatstroke in the current region, and he/she can take timely measures such as avoiding indoors and cooling down.

S303, outputting a notification that user's body temperature is abnormal through the processor when the user's body temperature is abnormal.

If it is determined that the user's body temperature is abnormal, a notification that the user's body temperature is abnormal will be send, which may be a voice notification to indicate whether the user's body temperature is too high or too low. It may also remind the user of the health risks at this time, as well as the ways to eliminate abnormal body temperature, and prompt the user to seek medical treatment in a timely manner. The sending manner may also be a light notification, which can indicate whether the user's body temperature is too high or too low through different light colors.

Further, the body temperature values of a preset user and/or the UV values in the environment, as well as the notification triggered by the body temperature values of the preset user and/or the UV values in the environment, can be sent to the preset client, allowing the user to view the body temperature values of the preset user and/or UV values in the environment and the notification through the client. The preset user includes the user and/or his/her associated users.

Among them, the preset user may include the associated users of the user, such as the user's guardian, specifically family members of the children and elderly people, as well as other personnel within the scope of responsibilities such as patients.

The preset client is a client set up on a smart mobile terminal used by the user. The user can view the body temperature values of the preset user's and/or the UV values in the environment through this client, as well as the notification triggered when the body temperature of the preset user's and/or the UV values in the environment are abnormal. For example, when the body temperature value is too high and reaches an abnormal level, the notification is to indicate that the preset user's body temperature is too high, as well as cooling measures that need to be taken. When the body temperature reach is too low and reaches an abnormal level, the notification is that the preset user's body temperature is too low, as well as heating measures need to be taken. For example, when the UV value is too high and reaches an abnormal level, the notification is that the preset user is in an environment where the UV intensity is too high, there is a risk of sunburn and entering the room. When the UV intensity is too low and reaches an abnormal level, the notification is that the preset user is in an environment where the UV intensity is too low and the exposure to UV is insufficient, measures such as increasing exposure to sunlight and outdoor activities are needed. Thus, remote access to health information notification for the preset user is achieved, which further improves the intelligence of health information notification.

In this embodiment of the present application, obtaining body temperature data measured by the temperature sensor provided on the glasses body of the smart glasses, determining whether user's body temperature is abnormal based on the body temperature data, and outputting relevant notification that the body temperature is abnormal if it is abnormal. The body temperature data measured by the temperature sensor on the glasses body is not easily affected by external factors, and the measurement accuracy of body temperature data is high, thereby improving the accuracy of determining user's body temperature abnormality, and thus improving the accuracy of indicating abnormal body temperature.

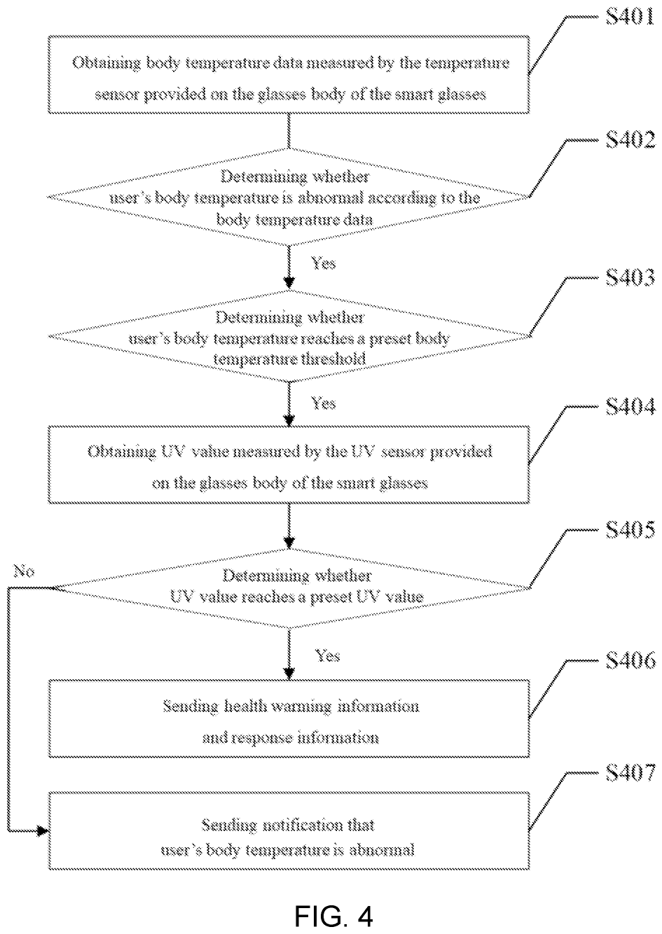

Referring to FIG. 4, which is a flowchart of a method for health information notification provided by another embodiment of this application. The method for health information notification is applied to the smart glasses of the above embodiments. The smart glasses include a glasses body, and temperature sensors, UV sensors and a processor provided on the glasses body. The processor is electrically coupled with the temperature sensors and the UV sensors. An execution subject may be the processor of the smart glasses, and the method includes:

S401, obtaining body temperature data measured by the temperature sensors through the processor;

S402, determining whether user's body temperature is abnormal through the processor based on the body temperature data;

If the user's body temperature is abnormal, step S403 is executed to further determine if it is a high temperature anomaly.

S403, determining whether user's body temperature reaches a preset body temperature threshold through the processor;

The preset body temperature threshold is a higher body temperature value, such as the temperature value that the human body may reach due to heatstroke, and may be 38° C.

If the preset body temperature threshold is reached, step S404 is executed.

S404, obtaining UV data measured by the UV sensors through the processor;

S405, determining whether the UV values exceed a preset UV value;

If it exceeds the preset UV value, step S406 is executed; and if not exceeded, step S407 is executed.

S406, outputting health warning information and response information by the controlling of the processor;

Abnormal high body temperature and strong UV rays may easily lead to heatstroke. Send health warning information and response information to the user through voice or flashing. The warning information is to remind the user of the possibility of heatstroke, and the response message is to prompt the user to enter the room, rest in a cool place, and drink water.

S407, outputting notification that user's body temperature is abnormal by the controlling of the processor.

Details not described in this embodiment may refer to the descriptions of the foregoing embodiments.

In this embodiment of the present application, obtaining body temperature data measured by the temperature sensors set on the glasses body, determining whether user's body temperature is abnormal based on the body temperature data. If it is abnormal, determining whether user's body temperature reaches a preset body temperature threshold. If it is reached, obtaining UV data measured by the UV sensors set on the glasses body, and determining whether the UV values exceed a preset UV value. If it exceeds, outputting the health warning information and response information, reminding the user that he/she may suffer from heatstroke. Due to the fact that the data of the temperature sensors and UV sensors on the glasses body are not easily affected by external factors, the measurement accuracy of body temperature data and UV data is high. Therefore, it improves the accuracy of determining whether the user's body temperature is abnormal and whether the UV rays are strong, thereby improving the accuracy of reminding users that they may experience heatstroke.

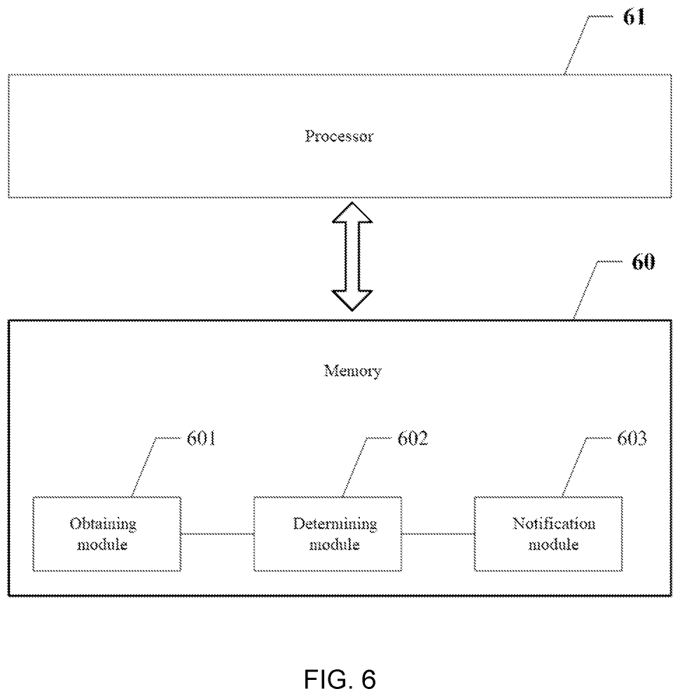

Referring to FIG. 6, which is a schematic view of a device for health information notification provided by an embodiment of this application. The device for health information notification may be a processor of the smart glasses or a unit within the processor. For easy of description, only relevant parts of this embodiment of the present application are shown. The device for health information notification includes a memory 60 storing computer programs thereon and a processor 61 coupled with the memory 60, wherein the processor 61 is used to call the computer programs stored on the memory 60 and execute them. The computer program includes:
acquisition module 601, for obtaining body temperature data measured by the temperature sensors provided on the glasses body of the smart glasses;
judgment module 602, for determining whether user's body temperature is abnormal based on the body temperature data;
notification module 603, for sending a notification that user's body temperature is abnormal when it is determined that the body temperature is abnormal.

Furthermore, the judgment module 602 is further used to count all of the measured body temperature data within the preset period, remove the body temperature data that is higher than the preset maximum limit or lower than the preset minimum limit, and calculate the average value of the remaining body temperature data. If the average value exceeds the preset normal range, it is confirmed that the user's body temperature is abnormal. Alternatively, the judgment module 602 counts all of the measured body temperature data within the preset period. If the number of times it exceeds the preset normal range is greater than the preset number, it is confirmed that the user's body temperature is abnormal.

Furthermore, the device for health information notification further includes a processing module;

The processing module is used to generate and save the corresponding relationship curve between the indoor temperature values and the measurement time based on the indoor temperature values by measured the temperature sensor and the measurement time;

When the indoor temperature value measured by the temperature sensor continues to rise, the increased indoor temperature value and measurement time will be compared with the corresponding relationship curve;

If the increased indoor temperature value and measurement time match the corresponding relationship curve, the user's current true body temperature data is determined based on the corresponding relationship curve;

If the increased indoor temperature value and measurement time do not match the corresponding relationship curve, the current measured body temperature data of the user will be used as the actual body temperature data of the user.

Further, the acquisition module 601 is further used to obtain the ambient temperature value measured by an external temperature sensor provided out of the glasses body;

The judgment module 602 is further used to compare this ambient temperature value with the ambient temperature value measured by the temperature sensor set on the glasses body. If the comparison result is inconsistent, the ambient temperature value measured by the external temperature sensor will be used as the true ambient temperature value.

The judgment module 602 is further used to determine whether user's body temperature reaches the preset body temperature threshold when the user's body temperature is abnormal;

The acquisition module 601 is further used to obtain the UV value measured by the UV sensor set on the glasses body of the smart glasses when it is reached the preset body temperature threshold;

The judging module 602 is further used to determine whether the UV value exceeds the preset UV value;

If so, the notification module 603 is further used to output health warning information and response information when the UV value exceeds the preset UV value.

The notification module 603 is further used to upload the user's body temperature values and/or UV values in the environment to the server, so that the server can send the current body temperature values and historical body temperature values of the user, and/or the current UV values and historical UV values in the environment to the client for display based on the user's query operation through the client.

The notification module 603 is further used to upload the user's body temperature values and/or UV values in the environment to the server, so that the server can analyze whether the uploaded body temperature values and/or UV values exceed the preset first body temperature reference value and/or preset first UV reference value respectively within the preset first analysis cycle, or, whether they exceed the preset second body temperature reference value and/or preset second UV reference value within the preset second analysis cycle. The first body temperature reference value is greater than the second body temperature reference value, and the first UV reference value is greater than the second UV reference value. Within the preset first analysis cycle, if the body temperature data exceeds the preset first body temperature reference value, and/or, if the UV value exceeds the preset first UV reference value; or, within the preset second analysis cycle, if the body temperature data exceeds the preset second body temperature reference value, and/or, if the UV value exceeds the preset second UV reference value, health warning information and response information will be sent to the client;

The notification module 603 is further used to confirm the current location of the user, upload the measured body temperature values and/or UV values in the environment to the server, so that the server can use the preset big data analysis method to determine that: among the preset population in the region, whether the number of people whose body temperature exceeds the preset body temperature warning value exceeds the preset number, and/or, whether the number of people whose UV value exceeds the preset UV warning value exceed the preset number of people. Among the preset population in the region, when the number of people whose body temperature exceeds the preset temperature warning value exceeds the preset number, and/or, if the number of people whose UV value exceeds the preset UV warning value exceeds the preset number, health warning information and response information will be sent to the client.

The notification module 603 is further used to send the preset user's temperature value and/or UV value in the environment, as well as the notification triggered when the preset user's temperature value and/or UV value in the environment are abnormal, to the preset client, so that the user can view the preset user's temperature value and/or UV value in the environment, as well as the notification through the client. The preset user includes the user's associated users.

Details not described in this embodiment may refer to the descriptions of the foregoing embodiments.

In this embodiment of the present application, obtaining body temperature data measured by the temperature sensor provided on the glasses body, determining whether user's body temperature is abnormal based on the body temperature data. If it is abnormal, determining whether user's body temperature reaches the preset body temperature threshold. If it is reached, obtaining UV data measured by the UV sensors provided on the glasses body, and determining whether the UV value exceeds the preset UV value. If it exceeds, outputting the health warning information and response information, reminding the user that he/she may suffer from heatstroke. Due to the fact that the data of the temperature sensors and UV sensors on the glasses body are not easily affected by external factors, the measurement accuracy of body temperature data and UV data is high. Therefore, it improves the accuracy of determining whether the user's body temperature is abnormal and whether the UV rays are strong, thereby improving the accuracy of reminding users that they may experience heatstroke.

Furthermore, an embodiment of this application further provides a computer-readable storage medium, which may be set in the smart glasses of the foregoing embodiments, or may be the memory in the smart glasses. A computer program is stored on the computer-readable storage medium, and is executed by the processor to realize the method for health information notification described in the foregoing embodiments shown in FIGS. 3 and 4. Further, the computer-storable storage medium may further be a U disk, a mobile hard disk, a read-only memory (ROM), a random-access memory (RAM), a magnetic disk or an optical disk, and other various media that may store the program code.

It should be noted that for the foregoing method embodiments, for simplicity of description, they are all expressed as a series of action combinations, but those skilled in the art should appreciate that the present application is not limited by the described sequence of actions. Because according to the present application, some steps may be performed in other sequences or simultaneously, and secondly, those skilled in the art should further appreciate that the embodiments described in the specification are all preferred embodiments, and the involved actions and modules are not necessarily all required by the present application.

In the above-mentioned embodiments, descriptions of the embodiment have particular emphasis respectively. For parts that are not described in detail in a certain embodiment, reference may be made to related descriptions of other embodiments.

The above is a description of the smart glasses, device and method for health information notification according to the present application. For those skilled in the art, according to the ideas of the embodiments of this application, changes may be made to specific implementations and application scopes. In summary, the content of this specification should not be construed as a limitation on the present application.

What is claimed is:

1. A pair of smart glasses, comprising:
a glasses body and a plurality of temperature sensors;
the temperature sensors being provided on an inner side of the glasses body, with a distance from a contact site of user's skin being less than a preset distance.

2. The smart glasses according to claim 1, wherein the temperature sensors are set at one or multiple positions of inner sides of temples of the glasses body, an inner side of an upper edge of a glasses frame of the glasses body, and an inner side of a lower edge of the glasses frame of the glasses body.

3. The smart glasses according to claim 1, wherein the temperature sensors are used to measure user's body temperature and/or ambient temperature around the user.

4. The smart glasses according to claim 1, wherein the smart glasses further comprise at least one UV sensor being set at one or multiple positions of an outer side of a glasses frame of the glasses body and outer sides of temples of the glasses body.

5. The smart glasses according to claim 4, wherein the at least one UV sensor comprises a plurality of UV sensors arranged on an upper face of the glasses frame.

6. The smart glasses according to claim 5, wherein a number of the plurality of UV sensors is two, and the two UV sensors are arranged at two lateral sides of the upper face of the front frame, respectively.

7. The smart glasses according to claim 1, wherein the smart glasses further comprise a plurality of external temperature sensors being set at one or multiple positions of an outer side of a glasses frame of the glasses body and outer sides of temples of the glasses body.

8. The smart glasses according to claim 7, wherein the external temperature sensors are used to measure an ambient temperature around the user.

9. A method for health information notification, applied to a pair of smart glasses which comprise a glasses body, and a temperature sensor and a processor provided on the glasses body, the processor being electrically coupled with the temperature sensor, the method comprising:
obtaining body temperature data measured by the temperature sensor through the processor;
determining whether user's body temperature is abnormal through the processor based on the body temperature data; and
outputting a notification that user's body temperature is abnormal through the processor when the user's body temperature is abnormal.

10. The method according to claim 9, wherein the step of determining whether user's body temperature is abnormal through the processor based on the body temperature data comprises:
counting all of the body temperature data measured during a preset period through the processor, removing body temperature data higher than a preset maximum limit or lower than a preset minimum limit, and calculating an average value of remaining body temperature data, wherein it is confirmed that user's body temperature is abnormal when the average value exceeds a preset normal range; or
counting all of the body temperature data measured during a preset period through the processor, wherein it is confirmed that user's body temperature is abnormal when a number of times that the body temperature data exceeds the preset normal range is greater than a preset number.

11. The method according to claim 10, further comprising:
obtaining ambient temperature value measured by an external temperature sensor set out of the glasses body through the processor, and comparing the ambient temperature value measured by the external temperature sensor with an ambient temperature value measured by the temperature sensor provided on the glasses body;
taking the ambient temperature value measured by the external temperature sensor as an actual ambient temperature value when a comparison result is inconsistent.

12. The method according to claim 10, wherein the smart glasses further comprise a UV sensor provided on the glasses body and electrically coupled with the processor, and the method further comprises:
determining whether user's body temperature reaches a preset body temperature threshold through the processor when the user's body temperature is abnormal;
obtaining UV data measured by the UV sensor through the processor when the user's body temperature reaches the preset body temperature threshold;
determining whether the UV value exceeds a preset UV value; and
outputting health warning information and response information by the controlling of the processor when the UV value exceeds the preset UV value.

13. The method according to claim 12, further comprising:
uploading user's body temperature values and/or UV values in the environment to a server through the processor, making the sever send current body temperature value and historical body temperature value of the user, and/or current UV value and historical UV value in the environment to the client for display based on user's query operation through the client; or
uploading user's body temperature values and/or UV values in the environment to the server through the processor, making the sever respectively analyze whether the uploaded body temperature values within a preset first analysis cycle exceed a preset first body temperature reference value, and/or, whether the uploaded UV values within the preset first analysis cycle exceed a preset first UV reference value; or, whether the uploaded body temperature values within a preset second analysis cycle exceed a preset second body temperature reference value, and/or, whether the uploaded UV values within the preset second analysis cycle exceed a preset second UV reference value, wherein the first body temperature reference value is greater than the second body temperature reference value, and the first UV reference value is greater than the second UV reference value;
sending the health warning information and response information to the client when, in the first analysis cycle, the body temperature values exceed the preset first body temperature reference value, and/or, the UV values exceed the preset first UV reference value; or, when, in the second analysis cycle, the body temperature values exceed the preset second body temperature reference value, and/or, if the UV values exceed the preset second UV reference value.

14. The method according to claim 12, further comprising:
confirming user's current location through the processor and uploading the measured user's body temperature values and/or UV values in the environment to a server, making the server use a preset big data analysis method to determine that: among the preset population in the region, whether a number of people whose body temperature exceeds a preset body temperature warning value exceeds a preset number of people, and/or, whether a number of people whose UV value exceeds a preset UV warning value exceeds a preset number of people;
sending the health warning information and response information to the client when, among the preset population in the region, the number of people whose body temperature exceeds the preset body temperature warning value exceeds the preset number of people, and/or, the number of people whose UV value exceeds the preset UV warning value exceeds the preset number of people.

15. The method according to claim 12, wherein the method further comprises:
sending the body temperature value of a preset user and/or UV value in the environment, as well as the notification triggered when the body temperature value of the preset user and/or UV value in the environment are abnormal to a preset client through the processer, making the user view the body temperature value of the preset user and/or UV value in the environment, as well as the notification through the client, wherein the preset user comprises the user's associated users.

16. A device for health information notification, comprising:
a memory for storing computer programs; and
a processor coupled with the memory, the processor being configured for calling and executing the computer programs stored on the memory;
the computer programs comprising:
an acquisition instruction for obtaining body temperature data measured by a temperature sensor set on a glasses body of a pair of smart glasses;
a judgment instruction for determining whether user's body temperature is abnormal based on the body temperature data;
a notification instruction for sending a notification that user's body temperature is abnormal when the user's body temperature is determined to be abnormal.

17. The device according to claim 16, wherein the judgment instruction is further used to:
count all of the body temperature data measured within a preset period, remove body temperature data higher than a preset maximum limit value or lower than a preset minimum limit value, and calculate an average value of remaining body temperature data, wherein it is confirmed that user's body temperature is abnormal when the average value exceeds a preset normal range; or
count all of the body temperature data measured within the preset period, wherein it is confirmed that user's body temperature is abnormal when a number of times that the body temperature data exceeds the preset normal range is greater than a preset number.

18. The device according to claim 17, wherein the acquisition instruction is further used to obtain ambient temperature value measured by an external temperature sensor set out of the glasses body;
the judgment instruction is further used to compare the ambient temperature value measured by the external temperature sensor with an ambient temperature value measured by the temperature sensor set on the glasses body;
the ambient temperature value measured by the external temperature sensor is taken as an actual environmental temperature value when the comparison result is inconsistent.

19. The device according to claim 17, wherein the judgment instruction is further used to determine whether user's body temperature reaches a preset body temperature threshold when the user's body temperature is abnormal;
the acquisition instruction is further used to obtain UV value measured by an UV sensor set on the glasses body of the smart glasses when the user's body temperature reaches the preset body temperature threshold;
the judgment instruction is further used to determine whether the UV value exceeds a preset UV value;
the notification instruction is further used to send health warning information and response information when the UV value exceeds the preset UV value.

20. The device according to claim 18, wherein the notification instruction is used to upload user's body temperature value and/or UV value in the environment to a server, making the server send the current body temperature values and historical body temperature values of the user, and/or the current UV values and historical UV values in the environment to the client for display based on the user's query operation through the client;
the notification instruction module is further used to upload the user's body temperature values and/or UV values in the environment to the server, making the server analyze whether the uploaded body temperature values within a preset first analysis cycle exceed a preset first body temperature reference value, and/or, whether the uploaded UV values within the preset first analysis cycle exceed a preset first UV reference value; or, whether the uploaded body temperature values within a preset second analysis cycle exceed a preset second body temperature reference value, and/or, whether the uploaded UV values within the preset second analysis cycle exceed a preset second UV reference value, wherein the first body temperature reference value is greater than the second body temperature reference value, and the first UV reference value is greater than the second UV reference value, and health warning information and response information are send to the client when, in the first analysis cycle, the body temperature value exceed the preset first body temperature reference value, and/or, the UV value exceed the preset first UV reference value; or, when, in the second analysis cycle, the body temperature value exceed the preset second body temperature reference value, and/or, the UV value exceed the preset second UV reference value;
the notification instruction module is further used to confirm user's current location and upload the measured user's body temperature values and/or UV values in the environment to the server, making the server use a preset big data analysis method to determine that: among the preset population in the region, whether a number of people whose body temperature exceeds a preset body temperature warning value exceeds a preset number, and/or, whether a number of people whose UV value exceeds a preset UV warning value exceeds a preset number, and the health warning information and response information are send to the client when, among the preset population in the region, the number of people whose body temperature exceeds the preset body temperature warning value exceeds the preset number, and/or, the number of people whose UV value exceeds the preset UV warning value exceeds the preset number;
the notification instruction is further used to send the body temperature value of a preset user and/or UV value in the environment, as well as the notification triggered when the body temperature value of the preset user and/or UV value in the environment are abnormal, to the preset client, making the user view the body temperature value of the preset user and/or UV value in the environment, as well as the notification through the client, wherein the preset user includes the user's associated users.
